(19) United States
(12) Patent Application Publication
(10) Pub. No.: US 2010/0119602 A1
(43) Pub. Date: May 13, 2010

(54) PHOSPHATE BINDER WITH REDUCED PILL BURDEN

(75) Inventors: Rudi E. Moerck, San Antonio, TX (US); Alan J. Gotcher, Incline Village, NV (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: SPECTRUM PHARMACEUTICALS, INC., Irvine, CA (US)

(21) Appl. No.: 12/640,339

(22) Filed: Dec. 17, 2009

Related U.S. Application Data
(63) Continuation of application No. 11/250,248, filed on Oct. 13, 2005.
(60) Provisional application No. 60/619,045, filed on Oct. 15, 2004.

(51) Int. Cl. A61K 9/36; A61K 33/24

(52) U.S. Cl. 424/480; 424/617

(57) ABSTRACT

The present invention is generally directed to compositions and formulations that can be used for the treatment of diseases such as End Stage Renal Disease (“ESRD”) and Chronic Renal Insufficiency (“CRI”). Specifically, it is directed to lanthanum-based compounds that bind phosphate and that can be formulated to provide for a reduced pill burden relative to other phosphate binders. In a formulation aspect of the present invention, a formulation is provided the includes a lanthanum-based, phosphate binder. The formulation is typically characterized in that in may be swallowed without chewing. Formulations of the present invention, along with a lanthanum-based compound, may optionally include the following: mass diluting agents; binders; coatings; compression/encapsulation aids; disintegrants; lubricants; plasticizers; slip/anti-electrostatic agents; powder lubricants; and, sweeteners. Where the formulation is in the form of a tablet, it typically has a volume between 0.3 cm³ and 1.2 cm³, preferably between 0.35 cm³ and 0.50 cm³. Each tablet typically includes enough phosphate binder such that only 3 or less tablets need to be ingested each day for a patient suffering from ESRD.

### PRIORITY CLAIM

This application claims priority to U.S. Provisional Patent Application Ser. No. 60/619,045, filed on Oct. 15, 2004, the entire disclosure of which is incorporated by reference.

### FIELD OF THE INVENTION

The present invention is generally directed to compositions and formulations that can be used for the treatment of diseases such as End Stage Renal Disease (“ESRD”) and Chronic Renal Insufficiency (“CRI”). Specifically, it is directed to lanthanum-based compounds that bind phosphate and that can be formulated to provide for a reduced pill burden relative to other phosphate binders.

### BACKGROUND OF THE INVENTION

In patients with ESRD, and to a lesser degree CRI, the kidneys are no longer capable of efficiently filtering and excreting wastes, resulting in increased concentrations of toxins and salts in the blood. ESRD patients normally require kidney dialysis to purify the blood of these unwanted materials. Phosphate normally enters a patient's serum by the ingestion of foods containing phosphates, which are very common in modern processed foods. Although phosphate is removed from a patient's blood, to some extent, during the kidney dialysis procedure, most kidney dialysis patients are not on daily dialysis and therefore require constant treatment for high levels of serum phosphate. In addition, CRI patients, who typically have not yet begun dialysis, may nevertheless require phosphate binder therapy as well, according to the current U.S. National Kidney Foundation K/DOQI guidelines. Elevated phosphate levels are not only hazardous because they can lead to weakening of the bones and hardening of the arteries, but they are also independently associated with increased mortality on dialysis.

When circulating phosphate levels are high, calcium and phosphate combine to lower the production of vitamin D and activate parathyroid hormone to release calcium from the bones (a condition known as secondary hyperparathyroidism). If left untreated, this condition can result in renal osteodystrophy, which is similar to osteoporosis, and is frequently associated with significant bone disease, fractures, and bone pain. High calcium phosphate levels can also lead to Cardiovascular Disease (“CVD”), which is associated with atherosclerosis.

To minimize the risk of elevated serum phosphate levels, ESRD and CKD patients must restrict dietary phosphorus intake and use oral phosphate-binding drugs to reduce absorption of phosphate from the gastrointestinal tract. Calcium-based phosphate binders have largely replaced aluminum-based phosphate binders which have been associated with significant toxic adverse effects, including dementia. However, use of calcium-based phosphate binders has evidenced negative side effects as well, including hypercalcemia and long-term progressive cardiovascular and soft tissue calcification.

Current prescription phosphate binders include: Nabi Pharmaceuticals' PHOSLO® (calcium acetate); Genzyme's RENAGEL® (sevelamer hydrochloride), the only non-calcium based drug for phosphate control approved by the FDA; and, Shire Pharmaceuticals' FOSRENOL® (lanthanum carbonate tetra hydrate, or “LCTH”).

### SUMMARY OF THE INVENTION

The present invention is generally directed to compositions and formulations that can be used for the treatment of diseases such as End Stage Renal Disease (“ESRD”) and Chronic Renal Insufficiency (“CRI”). Specifically, it is directed to lanthanum-based compounds that bind phosphate and that can be formulated to provide for a reduced pill burden relative to other phosphate binders.

In a formulation aspect of the present invention, a formulation is provided the includes a lanthanum-based, phosphate binder. The formulation is typically characterized in that in may be swallowed without chewing.

Formulations of the present invention, along with a lanthanum-based compound, may optionally include the following: mass diluting agents; binders; coatings; compression/encapsulation aids; disintegrants; lubricants; plasticizers; slip/anti-electrostatic agents; powder lubricants; and, sweeteners. Where the formulation is in the form of a tablet, it typically has a volume between 0.3 cm³ and 1.2 cm³, preferably between 0.35 cm³ and 0.50 cm³. Each tablet typically includes enough phosphate binder such that only 3 or less tablets need to be ingested each day for a patient suffering from ESRD.

In a method aspect of the present invention, a method of treating a patient is provided. The method involves administering a formulation of the present invention to a patient who has ESRD, CRI, Stage 3, or Stage 4 CKD.

### BRIEF DESCRIPTION OF THE DRAWINGS

FIG. 1 shows an X-ray diffraction scan of a compound made according to Example 1, as compared to a reference standard.

FIG. 2 shows an X-ray diffraction scan of a compound made according to Example 2, as compared to a reference standard.

### DETAILED DESCRIPTION OF THE INVENTION

The present invention is generally directed to compositions and formulations that can be used for the treatment of diseases such as End Stage Renal Disease (“ESRD”) and Chronic Renal Insufficiency (“CRI”). Specifically, it is directed to lanthanum-based compounds that bind phosphate and that can be formulated to provide for a reduced pill burden relative to other phosphate binders.

The lanthanum-based compounds can be considered platform drug candidates. The drug candidates target the treatment of elevated serum phosphate levels (i.e., hyperphosphatemia) in patenties with Stage 5 CKD, also known as ESRD, on dialysis, as well as patients with CRI that have not yet started dialysis (Stage 3 and Stage 4 CKD). Test results indicate that the lanthanum-based compounds have the ability to effectively control serum phosphate levels in CKD patients using significantly fewer grams of drug and pills per day as compared with other existing phosphate binding drugs. The compounds improve the treatment of hyperphosphatemia by decreasing the size of the phosphate binder dosage form and lowering the total daily pill burden of CKD patients.

The lanthanum-based compounds of the present invention are novel, second-generation candidates that have demonstrated effective phosphate removal and binding capacity in laboratory and animal testing. Animal testing of the compounds has been conducted, directly comparing the candidates to sevelamer hydrochloride (RENAGEL®) and LCTH (FOSRENOL®). The tests show improved performance in terms of dosage amount, tablet size, tolerance, adverse events, and lower cost as compared to competing products.

The compositions and formulations of the present invention set new performance standards and improve patient compliance through the following advantages over existing phosphate control drugs currently on the market: eased pill burden (lower number of tablets per meal and per day to bind an equal amount of phosphate); faster or equal serum phosphate titration; smaller, easier-to-swallow tablets; fewer adverse side effects; and, pricing flexibility. As patient compliance increases, usage of phosphate binders with the above characteristics will be favored both by patients and prescribing physicians. This will fuel market share gains for the present invention.

Increasing evidence indicates that some of the adverse outcomes of CKD can be prevented or delayed by early detection and treatment. Recent studies indicate potential therapeutic value of phosphate control for Stage 3 and Stage 4 CKD patients who exhibit a moderate to severe decline in kidney function. These studies have resulted in revised K/DQOI treatment guidelines, recently published by the U.S. National Kidney Foundation, a well-respected organization in the nephrology community. These guidelines are changing the way doctors approach early stage kidney disease by emphasizing the importance of identification and treatment of elevated phosphate earlier in the progression of the disease. Physicians are now beginning to prescribe phosphate-binding therapy for the approximate 8 million Stage 3 and Stage 4 CKD patients in the U.S.

The U.S. National Kidney foundation guidelines discourage the use of aluminum-based and magnesium-based phosphate binders in CKD patients. Aluminum is permissible only for very short-term use. Calcium-based phosphate binders are not recommended at all for patients at risk of hypercalcemia or that are already hypercalcemic. A growing trend in the treatment of hyperphosphatemia is the need for nephrologists to separate the treatment of serum calcium imbalances from the treatment of serum phosphate levels, suggesting an opportunity for more rapid growth of non-calcium based phosphate binders as compared with the overall phosphate binder market. The compositions and formulations of the present invention should allow nephrologists to separate the treatment of serum calcium imbalances from the treatment of serum phosphate levels.

In the U.S., Medicaid is expected to initiate coverage of oral medications in 2006. The compositions and formulations of the present invention, likely in an oral tablet form, may be covered by this planned change in payments. In addition to improving overall patient compliance, this should significantly increase the sales of all high-performance phosphate binders and decrease the use of over-the-counter phosphate binders, such as MYLANTA® or TUMS® for phosphate control.

The dosage requirement of compositions and formulations of the present invention is the lowest in its therapeutic class. Repeat prescriptions among patients using RENAGEL® are currently limited and have not met expectations due to high dosing and relatively high incidences of negative side effects. Data support the fact that the compositions and formulations of the present invention can effectively address these problems. Animal testing indicates that patients will require between 1.0 and 2.1 grams of the lanthanum-based compounds of the present invention, divided into 2 or 3 doses with meals, to initially achieve serum phosphate control comparable to that of RENAGEL® or FOSRENOL®. This is based on RENAGEL's® FDA-approved label and Shire's approved Swedish label for FOSRENOL®. The maintenance dosage of a lanthanum compound as used in the present invention is typically in the range of one 300 mg to 998 mg tablet, also taken two or three times per day. In contrast, patients taking RENAGEL® must keep track of and use 6 to 12 or more tablets per day, which equates to between 4.8 and 9.6 grams per day. Clinical trials suggest a dosage requirement of between 2.9 and 5.7 grams per day of FOSRENOL®. Furthermore, the compositions of the present invention can be formulated as an easy-to-swallow tablet.

The compositions and formulations of the present invention exhibit lower adverse events than competing products. Aluminum-based and calcium-based drugs raise concerns about potentially high incidences of adverse effects related to neurotoxicity and calcification of the eyes, soft tissue and coronary arteries, respectively, which generally limits their use. In addition RENAGEL® has shown a myriad of less serious but irritating side effects, including nausea, constipation, diarrhea, gas, bloating and increased acidity, resulting in heartburn and indigestion. Lanthanum-based compounds such as the ones used in the present invention and FOSRENOL® have demonstrated less common occurrences of these types of adverse events. Lanthanum-based compounds have also shown a high affinity for binding specifically to phosphate, which reduces the amount of binding with unwanted species and eliminates the need for additional treatments, such as vitamin therapy, often required for patients using RENAGEL®. FOSRENOL® and the lanthanum-based compounds of the present invention have low systemic absorption levels of lanthanum as well, according to results from animal testing in dogs. Testing further demonstrated that the compositions and formulations of the present invention have more acid neutralization capacity as compared to other compounds, including RENAGEL® and FOSRENOL®, which should correlate with reduced acidity and lower occurrences of heartburn and indigestion.

Studies have shown that lower patient cost for phosphate binding drugs generally increases their use and overall patient compliance. There are optional regulatory strategies available for the compositions and formulations of the present invention that offer the opportunity to substantially reduce costs for development and regulatory approval in the U.S. and Europe. These strategies potentially offer savings of significant development and clinical trial costs and therefore allow a degree of flexibility in eventual pricing and marketing not typically encountered when introducing a new drug to the pharmaceutical market.

The compositions and formulations of the present invention offer companies that are looking to build or compliment and established franchise in renal care therapeutics the opportunity to expand their product portfolio with a high-performance phosphate-binding drug. The recent expansion of indications for phosphate-binding drugs, including the revised U.S. National Kidney Foundation K/DOQI guideline, to Stage 3 and 4 CKD, suggest that using compositions and formulations of the present invention may be important to a pharmaceutical company looking to sustain or build a nephrology franchise. As the medical indications expand for their use, phosphate-binding therapies are positioned to become one to the most prescribed medications for the kidney disease patient population.

The present invention provides, among other things, compositions, formulations, methods of making formulations, methods of treatment and methods of doing business.

The lanthanum-based compounds used in the compositions and formulations of the present invention are typically either lanthanum carbonate hydroxides or lanthanum oxycarbonates (e.g., lanthanum oxycarbonate 2 hydrate and lanthanum dioxycarbonate). Lanthanum carbonate hydroxides may be hydrated or anhydrous. A typical anhydrous lanthanum carbonate hydroxide is LaCO₃OH. Lanthanum oxycarbonates may be hydrated or anhydrous. A typical hydrated lanthanum oxycarbonate is La₂O(CO₃)₂.xH₂O, where 1≦x≦3; a typical anhydrous lanthanum oxycarbonate is La₂O₂CO₃. Such compounds are discussed in U.S. Pat. Appl. 2004161474, which is hereby incorporated-by-reference for all purposes.

At the physiological stomach pH, around 3.0, the lanthanum oxycarbonates or lanthanum carbonate hydroxides exhibit a phosphate binding capacity of at least 300 mg of phosphate per gram of lanthanum compound. Most desirably, the lanthanum oxycarbonates exhibit a phosphate binding capacity of at least 400 mg PO₄/g of lanthanum compound. At the physiological pH of the upper small intestine, around 8.0, the lanthanum oxycarbonates still bind as much as 20 mg phosphate/g lanthanum compound.

In a formulation aspect, the present invention provides a phosphate binding formulation that can be directly swallowed as opposed to being chewed and swallowed. The formulation typically does not include an aluminum-, magnesium- or calcium-based phosphate binder; typically a lanthanum-based phosphate binder, as described in the preceding paragraph, is included.

The formulation typically includes the phosphate binder in a concentration greater than 50 percent by weight. Oftentimes, the phosphate binder is included in a concentration greater than 60, 70, 80, 90,95, 97.5 or 99 percent by weight.

Formulations of the present invention, along with a lanthanum-based compound, may optionally include the following: mass diluting agents; binders; coatings; compression/encapsulation aids; disintegrants; lubricants; plasticizers; slip/anti-electrostatic agents; powder lubricants; and, sweeteners. Where a mass diluting agent is included in the formulation, it is typically present in an amount between 20 and 75 percent by weight. Nonlimiting examples of mass diluting agents include lactose, sorbitol, mannitol, calcium phosphate, calcium sulphate, dextrose, sucrose, palatinate (equimolar mixture of D-glucopyranoside, 1,6 mannitol and D-glucopyranoside 1,6 glucitol).

Nonlimiting examples of binders include carbopol, povidone, xanthan gum, acacia, tragacanth, starches, sodium alginate, and sugars. Coating agents, where included, are typically present in a trace amount by weight. Nonlimiting examples of coating agents include cellulose phthalate, cellulose acetate phthalate, ethylcellulose, gellan gum, maltodextrin, methacrylates, methylcellulose, microcrystalline cellulose and carrageenan, shellac, sucrose and polyvinyl derivatives.

Compression agents/encapsulation aids, where included, are typically present in an amount between 2 and 20 percent by weight. Nonlimiting examples of compression agents/encapsulation aids include: microcrystalline cellulose (e.g., AVICEL®); PVP of molecular weight 10,000 to 30,000; calcium carbonate; dextrose; fructose; fructose DC; honey DC; lactose anhydrate; lactose monohydrate; lactose and aspartame; lactose and cellulose; lactose and microcrystalline cellulose; maltodextrin; maltose DC; mannitol; microcrystalline cellulose and guar gum; microcrystalline cellulose and lactose; molasses DC; sorbitol, crystalline; starch DC; and, sucrose.

Disintegrants, where included, are typically present in an amount between 0.5 and 15 percent by weight. Nonlimiting examples of disintegrants include: crosslinked vinylpyrrolidones (e.g., POLYCLAR AT®); crosslinked carboxymethylcelluloses; crosslinked croscarmelloses (e.g., ADDISOL®), carboxymethylamidons (e.g., AMIGEL®); crospovidone; gellan gum; L-HPC; sodium starch glycolate; and starch DC.

Lubricants, where included, are typically present in an amount between 0.1 and 3.0 percent by weight. Nonlimiting examples of lubricants include: glycerol palmitostearate, magnesium stearate; stearic acid; calcium stearate; alkaline stearate; talc; and, sodium stearyl fumarate.

Nonlimiting examples of plasticizers include: dibutyl sebacate; and, polyvinylacetate phthalate. Nonlimiting examples of powder lubricants include: glyceryl behenate.

Slip/anti-electrostatic agents, where included, are typically present in an amount between 0.1 and 2.0 percent by weight. Non-limiting examples of slip/anti-electrostatic agents include: colloidal silicas (e.g., AEROSIL® 100/200).

Nonlimiting examples of sweeteners include: aspartame; aspartame and lactose; dextrose; fructose DC; honey DC; maltodextrin; maltose DC; mannitol DC; molasses DC; sorbitol, crystalline; sorbitol, special solution; and, sucrose DC.

The formulation is typically in table form. Where a coating is used, it may be added, for example, to slow the disintegration of the table after administration (e.g., polymer coating) or to extend shelf life by shielding the tablet from picking up moisture.

Where ingredients other than a phosphate binder are included in the formulation, they are oftentimes included at a concentration less than 10 weight percent of the formulation, preferably less than 5 weight percent of the formulation.

A tablet of the present invention typically has a volume between 0.3 cm³ and 1.2 cm³, preferably between 0.35 cm³ and 0.50 cm³. Each tablet typically includes enough phosphate binder such that only 3 or less tablets need to be ingested each day for a patient suffering from ESRD or general kidney failure.

The tablet typically provides for rapid disintegration in the stomach after ingestion. Oftentimes, disintegration time in the stomach is less than 30 seconds. In certain cases, the disintegration time is less than 20 seconds or even 20 seconds.

The tablet typically exhibits a substantially longer shelf-life than other phosphate binding formulations. For instance, even after a period of 2 years, the tablet typically does not increase in volume more than 5 percent, preferably 2.5 percent, more preferably 1 percent.

In a method aspect of the present invention, a method of making the above-discussed formulation/tablet is provided. The method includes directly compressing a lanthanum-based phosphate binder using 1,000 to 50,000 lbs per square inch of pressure, preferably 2,000 to 6,000 lbs per square inch.

Other methods that can be used to produce the formulation/tablet include high shear mixer granulation (HSMG), fluidized bed granulation (FBG), and roll compaction (RC).

In another aspect, a method of treating a patient is provided. The method involves treating a patient who has ESRD or CRI with a composition or formulation of the present invention.

In another aspect, a further method of treating a patient is provided. The method involves treating a patient who has Stage 3 or Stage 4 CKD with a composition or formulation of the present invention (e.g., tablet).

In another aspect, a method of increasing patient compliance is provided. The method involves treating a patient who has ESRD, CRI, Stage 3 CKD or Stage 4 CKD with the composition/formulation of the present invention. Patient compliance is increased due to a number of factors, including eased pill burden, easy-to-swallow tablets and fewer adverse side effects. Typically patient compliance is increased at least 5 percent over that observed with RENAGEL® or FOSRENOL®. Oftentimes, patient compliance is increased at least 10 percent, 15 percent or even 20 percent.

In another aspect a method of doing business is provided. The method involves bringing the compositions or formulations of the present invention to market, resulting in a decrease of over-the-counter phosphate binders. Typically, sales of over-the-counter phosphate binders will be decreased at least 5 percent, preferably at least 10 or 15 percent.

In another aspect, a method of building or complimenting a franchise in renal care therapeutics is provided. The method involves acquiring the rights to market, sell or otherwise commercialize the compositions and formulations of the present invention.

### Examples

### Example 1

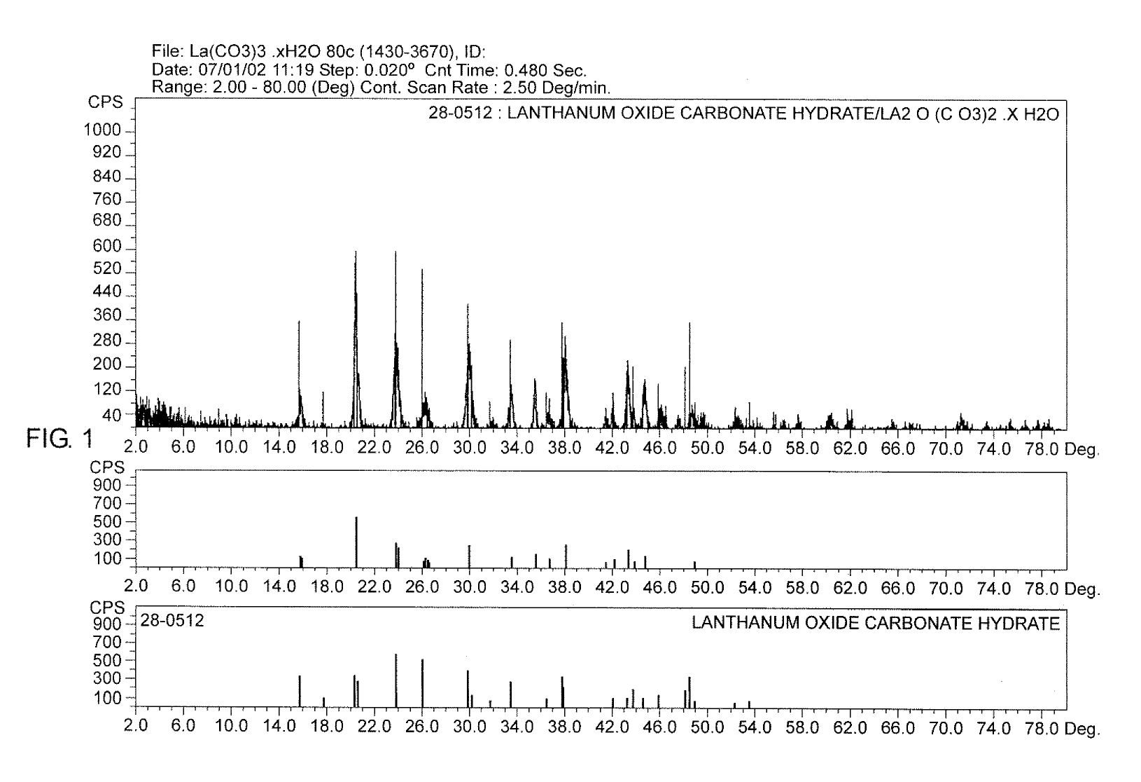

An aqueous HCl solution having a volume of 334.75 ml and containing LaCl₃ (lanthanum chloride) at a concentration of 29.2 wt % as La₂O₃ was added to a four liter beaker and heated to 80° C. with stirring. The initial pH of the LaCl₃ solution was 2.2. Two hundred and sixty five ml of an aqueous solution containing 63.59 g of sodium carbonate (Na₂CO₃) was metered into the heated beaker using a small pump at a steady flow rate for 2 hours. Using a Buchner filtering apparatus fitted with filter paper, the filtrate was separated from the white powder product. The filter cake was mixed four times with 2 liters of distilled water and filtered to wash away the NaCl formed during the reaction. The washed filter cake was placed into a convection oven set at 105° C. for 2 hours, or until a stable weight was observed. The product consists of lanthanum carbonate hydroxide, LaCO₃OH. FIG. 1 shows an X-ray diffraction scan of the compound as compared to a reference sample.

To determine the reactivity of the lanthanum compound with respect to phosphate, the following test was conducted. A stock solution containing 13.75 g/l of anhydrous Na₂HPO₄ and 8.5 g/l of HCl was prepared. The stock solution was adjusted to pH 3 by the addition of concentrated HCl. An amount of 100 ml of the stock solution was placed in a beaker with a stirring bar. Lanthanum oxycarbonate hydrate powder made as described above was added to the solution. The amount of lanthanum oxycarbonate hydrate powder was such that the amount of La in suspension was 3 times the stoichiometric amount needed to react completely with the phosphate. Samples of the suspension were taken at time intervals through a filter that separated all solids from the liquid. The liquid sample was analyzed for phosphorous.

### Example 2

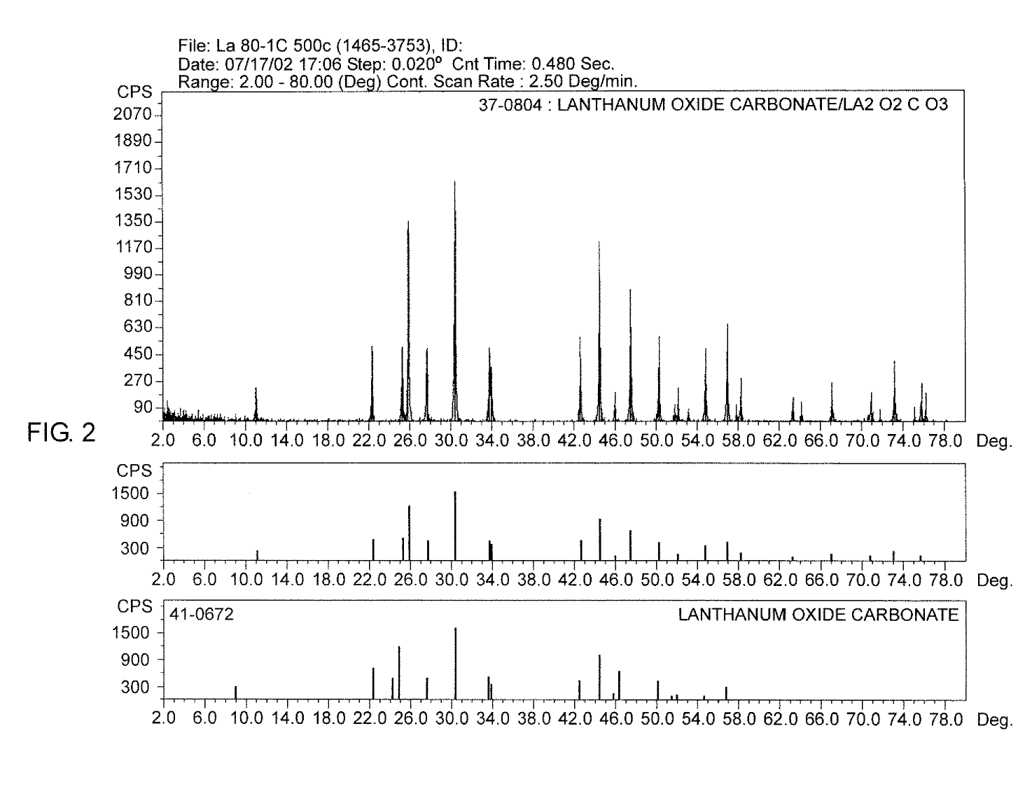

An aqueous HCl solution having a volume of 334.75 ml and containing LaCl₃ (lanthanum chloride) at a concentration of 29.2 wt % as La₂O₃ was added to a 4 liter beaker and heated to 80° C. with stirring. The initial pH of the LaCl₃ solution was 2.2. Two hundred and sixty five ml of an aqueous solution containing 63.59 g of sodium carbonate (Na₂CO₃) was metered into the heated beaker using a small pump at a steady flow rate for 2 hours. Using a Buchner filtering apparatus fitted with filter paper the filtrate was separated from the white powder product. The filter cake was mixed four times with 2 liters of distilled water and filtered to wash away the NaCl formed during the reaction. The washed filter cake was placed into a convection oven set at 105° C. for 2 hours until a stable weight was observed. Finally, the lanthanum oxycarbonate was placed in an alumina tray in a muffle furnace. The furnace temperature was ramped to 500° C. and held at that temperature for 3 hours. The resultant product was determined to be anhydrous lanthanum oxycarbonate La₂O₂CO₃. FIG. 2 shows an X-ray diffraction scan of the compound as compared to a reference standard.

The process was repeated three times. In one case, the surface area of the white powder was determined to be 26.95 m²/gm. A micrograph shows that the structure in this compound is made of equidimensional or approximately round particles of about 100 nm in size. An X-ray diffraction pattern showed that the product made is an anhydrous lanthanum oxycarbonate written as La₂O₂CO₃.

To determine the reactivity of this lanthanum compound with respect to phosphate, the following test was conducted. A stock solution containing 13.75 g/l of anhydrous Na₂HPO₄ and 8.5 g/l of HCl was prepared. The stock solution was adjusted to pH 3 by the addition of concentrated HCl. An amount of 100 ml of the stock solution was placed in a beaker with a stirring bar. Anhydrous lanthanum oxycarbonate made as described above, was added to the solution. The amount of anhydrous lanthanum oxycarbonate was such that the amount of La in suspension was 3 times the stoichiometric amount needed to react completely with the phosphate. Samples of the suspension were taken at intervals, through a filter that separated all solids from the liquid.

### Example 3

A solution containing 100 g/l of La as lanthanum acetate is injected in a spray-drier with an outlet temperature of 250° C. The intermediate product corresponding to the spray-drying step is recovered in a bag filter. This intermediate product is calcined at 600° C. for 4 hours. X-Ray diffraction of the product showed that it consists of anhydrous lanthanum oxycarbonate. The formula for this compound is written as (La₂CO₅).

To determine the reactivity of the lanthanum compound with respect to phosphate, the following test was conducted. A stock solution containing 13.75 g/l of anhydrous Na₂HPO₄ and 8.5 g/l of HCl was prepared. The stock solution was adjusted to pH 3 by the addition of concentrated HCl. An amount of 100 ml of the stock solution was placed in a beaker with a stirring bar. La₂CO₅ powder, made as described above, was added to the solution. The amount of lanthanum oxycarbonate was such that the amount of La in suspension was 3 times the stoichiometric amount needed to react completely with the phosphate. Samples of the suspension were taken at intervals through a filter that separated all solids from the liquid. The liquid sample was analyzed for phosphorous.

### Example 4

An aqueous HCl solution having a volume of 334.75 ml and containing LaCl₃ (lanthanum chloride) at a concentration of 29.2 wt % as La₂O₃ was added to a 4 liter beaker and heated to 80° C. with stirring. The initial pH of the LaCl₃ solution was 2.2. Two hundred and sixty five ml of an aqueous solution containing 63.59 g of sodium carbonate (Na₂CO₃) was metered into the heated beaker using a small pump at a steady flow rate for 2 hours. Using a Buchner filtering apparatus fitted with filter paper the filtrate was separated from the white powder product. The filter cake was mixed four times, each with 2 liters of distilled water and filtered to wash away the NaCl formed during the reaction. The washed filter cake was placed into a convection oven set at 105° C. for 2 hours or until a stable weight was observed. The X-Ray diffraction pattern of the product showed that it consists of lanthanum carbonate hydroxide, LaCO₃OH. The surface area of the product was determined by the BET method.

### Example 5

In Vivo Study in Rats

Groups of six adult Sprague-Dawley rats underwent ⅚th nephrectomy in two stages over a period of 2 weeks and were then allowed to recover for a further two weeks prior to being randomized for treatment. The groups received vehicle (0.5% w/v carboxymethyl cellulose), or lanthanum oxycarbonate suspended in vehicle, once daily for 14 days by oral lavage (10 ml/kg/day). The dose delivered 314 mg elemental lanthanum/kg/day. Dosing was carried out immediately before the dark (feeding) cycle on each day. Urine samples (24 hours) were collected prior to surgery, prior to the commencement of treatment, and twice weekly during the treatment period. Volume and phosphorus concentration were measured.

Feeding—During the acclimatization and surgery period, the animals were given Teklad phosphate sufficient diet (0.5% Ca, 0.3% P; Teklad No. TD85343), ad libitum. At the beginning of the treatment period, animals were pair fed based upon the average food consumption of the vehicle-treated animals the previous week.

⅚ Nephrectomy—After one week of acclimatization, all animals were subjected to ⅚ nephrectomy surgery. The surgery was performed in two stages. First, the two lower branches of the left renal artery were ligated. One week later, a right nephrectomy was performed. Prior to each surgery, animals were anesthetized with an intra-peritoneal injection of ketamine/xylazine mixture (Ketaject a 100 mg/ml and Xylaject at 20 mg/ml) administered at 10 ml/kg. After each surgery, 0.25 mg/kg Buprenorphine was administered for relief of post-surgical pain. After surgery, animals were allowed to stabilize for 2 weeks to beginning treatment.

Results show a decrease in phosphorus excretion, a marker of dietary phosphorus binding, after administration of the lanthanum oxycarbonate or lanthanum carbonate hydroxide (at time>0), compared to untreated rats.

### Example 6

Dog Study

Six adult beagle dogs were dosed orally with capsules of lanthanum oxycarbonate LaCO₃OH (compound A) or La₂O₂CO₃ (compound B) in a cross-over design using a dose of 2250 mg elemental lanthanum twice daily (6 hours apart). The doses were administered 30 minutes after provision of food to the animals. At least 14 days washout was allowed between the crossover arms. Plasma was obtained pre-dose and 1.5, 3, 6, 7.5, 9, 12, 24, 36, 48, 60, and 72 hours after dosing and analyzed for lanthanum using ICP-MS. Urine was collected by catheterization before and approximately 24 hours after dosing and creatinine and phosphorus concentrations measured. The tests led to reduction of urine phosphate excretion, a marker of phosphorous binding.

1. A formulation, wherein the formulation comprises a phosphate binder, and wherein the formulation has a volume between 0.3 cm³ and 1.2 cm³, and wherein the formulation includes enough phosphate binder such that only 3 or less tablets need to be ingested each day for a patient suffering from End Stage Renal Disease or general kidney failure.

2. The formulation according to claim 1, wherein the phosphate binder is selected from a group consisting of: LaCO₃OH; La₂O(CO₃)₂.xH₂O where 1≦x≦3; and, La₂O₂CO₃.

3. The formulation according to claim 1, wherein the formulation further comprises a mass diluting agent, and wherein the mass diluting agent is selected from a group consisting of: lactose, sorbitol, mannitol, calcium phosphate, calcium sulphate, dextrose, sucrose, and palatinate.

4. The formulation according to claim 1, wherein the formulation further comprises a binder, and wherein the binder is selected from a group consisting of: carbopol, povidone, xanthan gum, acacia, tragacanth, starches, sodium alginate, and sugars.

5. The formulation according to claim 1, wherein the formulation further comprises a coating, and wherein the coating is selected from a group consisting of: cellulose phthalate, cellulose acetate phthalate, ethylcellulose, gellan gum, maltodextrin, methacrylates, methylcellulose, microcrystalline cellulose and carrageenan, shellac, sucrose and polyvinyl derivatives.

6. The composition according to claim 1, wherein the composition further comprises a compression agent or encapsulation aid, and wherein the agent or aid is selected from a group consisting of: microcrystalline cellulose; PVP of molecular weight 10,000 to 30,000; calcium carbonate; dextrose; fructose; fructose DC; honey DC; lactose anhydrate; lactose monohydrate; lactose and aspartame; lactose and cellulose; lactose and microcrystalline cellulose; maltodextrin; maltose DC; mannitol; microcrystalline cellulose and guar gum; microcrystalline cellulose and lactose; molasses DC; sorbitol, crystalline; starch DC; and, sucrose.

7. The composition according to claim 1, wherein the composition further comprises a disintegrant, and wherein the disintegrant is selected from a group consisting of: crosslinked vinylpyrrolidones; crosslinked carboxymethylcelluloses; crosslinked croscarmelloses; carboxymethylamidons; crospovidone; gellan gum; L-HPC; sodium starch glycolate; and starch DC.

8. The composition according to claim 1, wherein the composition further comprises a lubricant, and wherein the lubricant is selected from a group consisting of: glycerol palmitostearate, magnesium stearate; stearic acid; calcium stearate; alkaline stearate; talc; and, sodium stearyl fumarate.

9. The composition according to claim 1, wherein the composition further comprises a plasticizer, and wherein the plasticizer is selected from a group consisting of: dibutyl sebacate; and, polyvinylacetate phthalate.

10. The composition according to claim 1, wherein the composition further comprises a powder lubricant, and wherein the powder lubricant is glyceryl behenate.

11. The composition according to claim 1, wherein the composition further comprises a slip or anti-electrostatic agent, and wherein the agent is a colloidal silica.

12. The composition according to claim 1, wherein the composition further comprises a sweetener, and wherein the sweetener is selected from a group consisting of: aspartame; aspartame and lactose; dextrose; fructose DC; honey DC; maltodextrin; maltose DC; mannitol DC; molasses DC; sorbitol, crystalline; sorbitol, special solution; and, sucrose DC.

13. A method of treating End Stage Renal Disease, wherein the method comprises administration of a formulation according to claim 1.

14. A method of treating End Stage Renal Disease, wherein the method comprises administration of a formulation according to claim 2.

15. A method of making a composition according to claim 1, wherein the method comprises directly compressing a lanthanum-based phosphate binder using 1,000 to 50,000 lbs per square inch of pressure.
